(19) United States
(12) Patent Application Publication
(10) Pub. No.: US 2020/0357523 A1
(43) Pub. Date: Nov. 12, 2020

(54) Early Feedback of Disease Factors to Improve Patient Quality of Life, Engagement and Persistence

(71) Applicant: Curelator, Inc., Palmer, MA (US)

(72) Inventors: Alec Mian, Palmer, MA (US); Marina Vives-Mestres, Palmer, MA (US); Takeichi Kanzaki Cabrera, Palmer, MA (US); Francisco Vazquez Barreiros, Palmer, MA (US)

(21) Appl. No.: 16/965,463

(22) PCT Filed: Jan. 31, 2019
(86) PCT No.: PCT/US2019/016094

§ 371 Date: Jul. 28, 2020

Related U.S. Application Data
(60) Provisional application No. 62/624,449, filed on Jan. 31, 2018.

(51) Int. Cl. G16H 50/30; G16H 10/20; G16H 50/70; G16H 10/60

(52) U.S. Cl. CPC G16H 50/30; G16H 10/60; G16H 50/70; G16H 10/20

(57) ABSTRACT

The disclosed systems and methods include receiving, individual tracking data corresponding to a patient, determining. Systems and methods further include, based on (i) the individual tracking data and (ii) aggregate tracking data received by the health management application from a population of patients, a list of no-association factors (NAFs) for the patient. Systems and methods further include determining that the patient has been exposed to a particular NAF a minimum threshold number of times without experiencing a particular disease symptom associated with the particular NAF. Systems and methods further include, providing patient feedback in response to determining that the patient has been exposed to the particular NAF the threshold number of times without experiencing the particular disease symptom, where providing the patient feedback includes providing an indication of the particular NAF.

### CROSS-REFERENCE TO RELATED APPLICATIONS

This application claims priority to U.S. provisional application 62/624,449 filed on Jan. 31, 2018, the entire content of which is incorporated herein by reference.

### BACKGROUND

Some current techniques for patients to manage disease symptoms include keeping a written record of times when disease symptoms occur as well as times when the patient engages in potential triggering (and perhaps mitigating) activities. Other current techniques may include a patient keeping an electronic diary of disease symptoms, disease triggering/mitigating activities, along with perhaps other disease monitoring and management related data, perhaps by way of an application. In instances where patients keep an electronic diary using an application, patients may exhibit varying levels of engagement.

### SUMMARY

Many digital health management applications require and/or rely at least in part on patient participation in inputting or otherwise providing patient data to the health management application so that the health management application can analyze the patient's data and provide the patient with meaningful information about the patient's health and healthcare. For example, some digital health management applications require patients to enter data into the health management application (e.g., via a user interface) and/or wear one or more data gathering devices that report data to the health management application (e.g., a fitness tracker, blood glucose monitor, or other device configured to track health-related data). As a result, and using the act of self-reporting data as a non-limiting example of participation, both the amount of time a particular patient is willing to spend each day entering data (“engagement”) and how many days the patient is willing to continue entering data (“persistence”) are very important to building a robust data set for that patient. Because most health management applications require some minimally sufficient data set to provide useful feedback to a patient, and because the risk that a patient will lose interest in a health management application and quit entering data is highest during the first few days to weeks after the patient first starts using the health management application, there is a risk that the patient will simply lose interest and cease entering data into the healthcare management application (and perhaps even quit using the application altogether) before the healthcare management application has acquired enough data to provide meaningful health and/or health related feedback to the patient. Thus, there is a need for systems and methods to increase patient engagement and patient persistence for digital health management applications.

This application discloses systems and methods for increasing patient engagement and patient persistence for a digital health management application. Some embodiments include selecting and providing patient-specific information of high interest and clinical use to the patient via a patient-interaction-based feedback schedule designed to encourage daily engagement and longitudinal persistence. In operation, the selection of patient-specific information of high interest and clinical use to the patient is based at least in part on aggregate patient data and individual patient data, including but not limited to individual patient data that the patient has very recently entered, thereby providing valuable feedback to the patient very soon after the patient first begins using the digital health management application, which encourages the patient to continue entering more patient data.

While encouraging daily engagement is a goal of some embodiments, other embodiments may be designed to additionally or alternatively encourage engagement over different timeframes, such as requiring the user to enter data only one day per week, or every day for one week every two months, or every day for one month every six months, or other schedules, depending on the type of patient data required or desired and the type of analysis performed by the healthcare management application.

As used herein, engagement generally refers to a degree to which a patient interacts with a health management application, including but not limited to one or more of an amount of time a patient spends interacting with the health management application, and including but not limited to engagement per hour, day, month, week, quarters, year, or any other timeframe and/or an amount of data the patient enters via the health management application each unit of time. In some embodiments, the inventive methods can flexibly comprise engagement schedules specific for each individual patient and can vary with the patient's schedule provided that the level of engagement is sufficient to provide patient-specific information of high interest and clinical use to the patient.

For example, engagement includes but is not limited to a patient's time per day using the health management application, the patient's time per session using the health management application, the amount of data the patient enters per day (or week, month, year, quarter, etc.), the amount of data the patient enters per session, the number of times (sessions) that a patient interacts with the health management application per day (or week, month, quarter, year, etc.), the amount of data the patient enters per session, and/or similar metrics that quantify a degree to which the patient is interacting with and/or inputting data into the health management application. Further, as used herein, persistence generally refers to engagement over time, including but not limited to engagement over days, months, weeks, quarters, years, or any other timeframe.

In some embodiments, a patient enters data (sometimes referred to herein as tracking data) relating to a particular disease or disorder into the health management application. The data includes but is not limited to: (i) data about a particular symptom of the disease or disorder, such as whether the patient experienced the disease symptom (including where relevant the frequency of the symptom's occurrence), when the patient experienced the symptom, where the patient experienced the symptom, the severity of the symptom, and so on; (ii) data about disease factors related to the disease or disorder; (iii) data about actual and/or suspected triggers associated with the disease or disorder; and/or (iv) other data relating to whether and the extent to which the patient has experienced a particular disease symptom, disease factor, disease trigger, and/or other data relevant to managing and/or monitoring a disease or disorder of the patient.

As used herein, a disease symptom is a physical manifestation of a particular disease or disorder. In operation, a disease symptom can be characterized by multiple characterization metrics, including but not limited to one or more of: (i) a time (or range of times) when the patient experienced the disease symptom; (ii) a severity of the disease symptom; (iii) aspects or characteristics describing the disease symptom; and/or (iv) whether the disease symptom was accompanied by other related disease symptoms (and perhaps disease factors and/or disease triggers as well). In non-limiting examples, where the disease is migraine and the disease symptom is a migraine headache, the characterization metrics for the migraine headache symptom may include any one or more of: (i) when the headache occurred; (ii) how long the headache lasted; (iii) the intensity and/or severity of the headache; (iv) the location of the headache along the patient's head; and/or (v) whether the headache was accompanied by other related symptoms such as nausea or dizziness, and if so, the time, duration, intensity/severity of the accompanying symptoms. Disease symptoms for other chronic diseases may include different characterization metrics.

As used herein, a disease factor is any event, exposure, action, or conduct related to and/or performed or experienced by a patient that has the potential to influence, affect, or cause the patient to experience a disease symptom, or in some cases, prevent the patient from experiencing a disease symptom. Disease factors may include both: (i) voluntary or modifiable conduct and/or experiences by the patient over which the patient has at least some control, such as emotional states (anger, boredom, stress, anxiety, etc.), consumption of a particular food product, ingestion of a particular therapeutic agent, application of a particular therapeutic agent, ingestion of a particular dietary supplement or drug, performance of a particular physical activity, and/or exposure to a particular chemical agent; and (ii) involuntary or un-modifiable conduct and/or experiences, such as exposure to environmental factors (e.g., smog, sunlight, rain, snow, high or low humidity, or high or low temperatures), ingestion or other exposure to mandatory therapeutic agents or drugs (e.g., drugs to treat or maintain other diseases), and effects of other diseases or physical conditions over which the patient has little or perhaps effectively no control.

Like a disease symptom, a disease factor can also be characterized by multiple characterization metrics, and different disease factors may have different characterization metrics. For example, for a food or drug consumption-based disease factor, the characterization metrics may include, for example: (i) when the patient consumed the food or drug; and/or (ii) how much of the food or drug the patient consumed. Characterization metrics for an exposure-based disease factor may include, for example: (i) when the patient was exposed; (ii) the intensity (e.g., bright sunlight) of the exposure; and/or (iii) the duration of the exposure.

In some embodiments, disease factors may also include premonitory symptoms or warning signs that may not actually cause the patient to experience a disease symptom but may just be closely associated with onset of a disease symptom for a particular patient. To use the migraine example again, a premonitory symptom might be a craving for sweet foods perhaps caused by a chemical change in the patient's body before the patient experiences the migraine. In this example, the sweet craving does not cause the migraine, but instead is likely caused by some chemical change that also causes the patient to experience the migraine.

In some instances, a particular physical manifestation felt by the patient may be a disease symptom or a disease factor depending on the context. To use diabetes as an example, abnormal body temperature, abnormal heart rate, and abnormal blood sugar levels may be disease factors because they tend to cause a disease symptom associated with diabetes. But in other contexts, abnormal body temperature, abnormal heart rate, and abnormal blood sugar levels may be disease symptoms that are caused by other disease factors.

As used herein, a disease trigger is a disease factor that has been determined, for example through statistical analyses or other methods, to have a sufficiently strong association with a particular disease symptom for an individual patient so as to become patient-specific information of high interest and clinical use to the patient. In some cases, a disease trigger may be strongly associated with causing the patient to experience the particular disease symptom, or at least increasing the risk or likelihood that the patient will experience the particular disease symptom. In other cases, a disease trigger may be strongly associated with preventing the patient from experiencing the particular disease symptom, or at least decreasing the risk or likelihood that the patient will experience the particular disease symptom; such disease triggers may be referred to herein as protectors because they tend to reduce the patient's likelihood of experiencing the disease symptom. In some embodiments, a disease trigger for a patient is a disease factor having a determined univariate association with a disease symptom for the patient, where the determined univariate association has a Cox Hazard Ratio greater than 1 and a p-value less than or equal to 0.05, or where the determined univariate association has a significantly greater than 0 coefficient (p-value less than or equal to 0.05) in a logistic regression or in equivalent multivariate models.

For the majority of patients, many commonly-suspected disease factors are not disease triggers, i.e., the most suspected disease factors are neither triggers nor protectors for the patient. However, there is a high degree of heterogeneity between patients with respect to which disease factors affect different patients. To help a patient manage his or her disease, the patient ideally wants individualized information about which suspected disease factors are disease triggers/protectors for them and which suspected disease factors have no effect for them. A suspected disease factor that has no effect for that individual is referred to herein as a “no-association factor” or NAF.

Knowing that a suspected disease factor is an NAF is valuable information for patients because an NAF affects the patient's quality of life but does not influence patient outcomes. For example, if a migraine patient believes chocolate (a suspected personal disease factor) increases his or her likelihood of experiencing a migraine headache (i.e., the patient believes chocolate is a migraine trigger), the patient may occasionally or consistently avoid chocolate and foods that contain chocolate. But if the patient becomes aware, for example by the practice of the methods disclosed herein, that chocolate is an NAF (in this example, chocolate may be referred to as a personal NAF), then the patient can enjoy chocolate without fear that consuming the chocolate will cause a migraine headache, thereby improving the patient's quality of life. Further, informing the patient that chocolate is an NAF is compatible with clinical trials of drugs, devices, and other therapeutic interventions because knowledge of NAFs and any subsequent behavior change as a result of this knowledge, uniquely and beneficially does not influence therapeutic efficacy of trials where engagement is required. Also, the mistaken belief that, e.g. chocolate, is a disease trigger when for that patient it is actually an NAF can produce a confounding variable for any assessment method for actual disease triggers for that patient, and are advantageously avoided.

Determining whether a particular disease factor is (or is not) a disease trigger or protector for an individual patient can take time because most algorithms for correlating disease factors with disease symptoms require a lot of data over time to reach a conclusion as to whether the disease factor is a disease trigger with a sufficiently high confidence. However, aggregate data from a large population of patients can provide a probability that a particular disease factor is an NAF for the patient even before the patient has entered sufficient data to produce an individualized conclusion for that patient. By combining individual tracking data received from the patient with the aggregate tracking data from the large population of patients, the health management application can identify some NAFs with relatively little patient input information and very early in the patient's engagement with the health management application, for example, even after the patient has entered tracking data confirming that the patient did not experience a disease symptom after even just one or two exposures to a suspected disease factor. Such positive results from minimal engagement can act as a potent incentive for the patient to continue to engage with the health management application.

As used herein, individual tracking data may refer to data entered by an individual patient into the health management application, and may include responses to questionnaires provided to the individual patient that solicit responses related to disease factors and disease symptoms of one or more diseases or disorders. As used herein, aggregate tracking data may refer to data entered by an a population of patients into the health management application, and may include responses to questionnaires provided to the population of patients that solicit responses related to disease factors and disease symptoms of one or more diseases or disorders. Disease factors may be referred to as population disease factors, personal disease factors, and global disease factors. As described above, some disease factors may be NAFs for one or more patient populations, one or more individual patients, or for everyone, and thus, NAFs may be referred to population NAFs, personal NAFs, and global NAFs. As used herein, population disease factors may refer to disease factors that are tracked by the health management application in connection with a particular disease based on aggregate tracking data received from a population of patients. As used herein, personal disease factors may refer to disease factors that are tracked by the health management application in connection with a particular disease based on individual tracking data received by the health management application and based on disease factors selected by a patient in connection with the particular disease or disorder. As used herein, global disease factors may refer to all disease factors that are tracked by the health management application in connection with a particular disease, including all population disease factors and all personal disease factors tracked by the health management application. Similarly, as used herein, population NAFs may refer to a subset of population disease factors that have no effect for an individual patient. As used herein, personal NAFs may refer to a subset of personal disease factors that have no effect for an individual patient. And, as used herein, global NAFs may refer to a subset of global disease factors that have no effect for an individual patient.

For example, if disease factor X is commonly suspected to be a disease trigger, but disease factor X has been determined to be an NAF for most patients in the aggregate population (e.g., based on all or substantially all past patient tracking data received from patients in the population), the health management application can conclude that disease factor X is an NAF for the patient after the patient has been exposed to disease factor X only once or twice with no subsequent disease attack. Without the historical aggregate data confirming that disease factor X is an NAF for the past patients, the health management application may require tracking data confirming that the patient has been exposed to disease factor X up to 10 or more times without experiencing a subsequent disease symptom or may require a minimum amount of experienced disease symptoms (for example, 8 experienced disease symptoms) and a minimum amount of variability in the disease factor X before there is sufficient statistical power for the health management application to determine that the disease factor X is an NAF with sufficient confidence. In this example, disease factor X may initially be referred to as a population disease factor. Because disease factor X is later determined to have no effect on the patient, disease factor X may be referred to as a population NAF.

Because the health management application can confirm that a particular disease factor is an NAF after the patient has entered relatively little tracking data (e.g., sometimes only 1 or 2 entries of the particular disease factor), the health management application can inform the patient of the NAF very soon after the patient first begins entering tracking data into the health management application. For example, the health management application may provide an indication of an NAF within a day or two days of first receiving individual tracking data. Doing so may, in examples, increase patient engagement with the health management application by providing relatively quick feedback. But rather than inform the patient immediately after determining that a particular disease factor is an NAF, the health management application can instead, at least in some embodiments, withhold the NAF determination and provide the NAF as a “reward” to the patient later.

For example, when the patient receives feedback from the health management application that disease factor Y is an NAF (and thus, the patient can be rewarded with the option of no longer needing to enter tracking data about disease factor Y), that feedback serves a reward of sorts to the patient because the feedback (i) confirms to the patient that entering the tracking data has generated a beneficial result (i.e., the patient now knows that disease factor Y has no effect on whether he or she will experience the disease symptom), and (ii) reduces the amount of tracking data that the patient needs to enter going forward (i.e., the patient now has one less disease factor to track via the healthcare management application).

In some embodiments, the health management application stores NAFs and informs the patient of the NAFs according to a reward schedule designed to increase patient interaction and persistence with the healthcare management application. In some embodiments, the health management application varies both the timing of the rewards (i.e., the interval between rewards varies) and the size of the reward (i.e., the number of disease factors confirmed as NAFs). For instance, the health management application can provide feedback according to an intermittent feedback schedule, in which providing confirmed NAFs to a patient is performed randomly, or randomly but subject to constraints. As an example of a constraint, the chances of providing an NAF at a given time might be 0% if there are no confirmed NAFs to provide. Further, in some embodiments, the variation in timing and size of the rewards is based on how often the patient has been interacting with the health management application and/or the quantity of tracking data the patient has been entering into the health management application.

In some embodiments, the health management application could also treat feedback confirming that a particular disease factor is a disease trigger as a reward and intersperse confirmed triggers with confirmed NAFs according to the reward schedule. But in preferred embodiments, the health management application informs the patient that a particular disease factor is a disease trigger (e.g., a trigger or protector) separately from any reward schedule so that the patient can start avoiding certain triggers (or perhaps seek out certain protectors) as soon as practicably possible so as to avoid experiencing a disease symptom.

In some embodiments, the health management application could also feedback disease factors that are shown not to vary (invariable factors, or “IFs”) across the self-reported individual tracking data that cannot be analyzed through any statistical method. For example, for a disease factor of monosodium glutamate (MSG) consumption, if a patient reports a constant “no consumption” of MSG, the healthcare management application could provide MSG consumption to the patient as an IF to (i) reduce the amount of tracking data that the patient needs to enter going forward or (ii) help the patient decide whether to start varying the exposure to the disease factor (e.g., decide whether to consume MSG) to be able to confirm whether the disease factor is a trigger, a protector, or an NAF.

In some embodiments, the disclosed systems and methods for increasing patient engagement and persistence are (or at least can be) used with health management applications that monitor and collect data about a patient's disease symptoms, disease factors, and/or disease triggers, aggregate and/or organize information regarding the patient's disease symptoms, disease factors, and/or disease triggers, and provide feedback relating to how the patient's conduct or environment contribute to the patient's near term likelihood of experiencing a particular disease symptom. For example, the disclosed systems and methods may be used with any of the health management applications disclosed and described in U.S. application Ser. No. 15/502,087 titled “Chronic Disease Discovery and Management System,” filed on Feb. 6, 2017, and which claims priority to (a) PCT application PCT/US15/43945 titled “Chronic Disease Discovery and Management System,” filed on Aug. 6, 2015, (b) U.S. provisional application 62/034,408 titled “Disease Symptom Trigger Map,” filed on Aug. 7, 2014; (c) U.S. provisional application 62/120,534 titled “Chronic Disease Management System,” filed on Feb. 25, 2015; (d) U.S. provisional application 62/139,291 titled “Chronic Disease Discovery and Management System,” filed on Mar. 27, 2015; (e) U.S. provisional application 62/148,130 titled “Chronic Disease Discovery and Management System,” filed on Apr. 15, 2015; and (f) U.S. provisional application 62/172,594 titled “Chronic Disease Discovery and Management System,” filed on Jun. 8, 2015. The systems and methods disclosed in the above-listed applications are practiced by identifying disease factors and triggers for a patient population, and (based on the identified disease factors and triggers for the patient population) identifying disease factors and triggers for an individual patient. Based on the patient's disease triggers and a strength of the association between the individual patient's experienced disease triggers and a disease symptom, these systems and methods may show the patient the impact that his or her conduct or environment might have on the patient's near term likelihood of experiencing the particular disease symptom. The above-listed applications are owned by Curelator, Inc., and this application incorporates the entire contents of the above-listed applications by reference.

Further, in some embodiments, the disclosed systems and methods for increasing patient engagement and persistence are (or at least can be) used with techniques for predicting correlations between patient actions and disease symptoms based on data collected from a sufficient plurality of individual patients, and in turn, predicting for an individual patient, whether performing a particular action is likely to improve a disease symptom for that particular patient. For example, the disclosed systems and methods may be used with any of the methods or systems disclosed and described in PCT application PCT/US14/13894 titled “Methods and Systems for Determining a Correlation Between Patient Actions and Symptoms of a Disease,” filed on Jan. 30, 2014, which claims priority to (a) U.S. provisional application 61/860,893 titled “Methods and Systems for Determining a Correlation Between Patient Actions and Symptoms of a Disease,” filed on Jul. 31, 2013; (b) U.S. provisional application 61/762,033 titled “Methods and Systems for Determining a Correlation Between Patient Actions and Symptoms of a Disease,” filed on Feb. 7, 2013; and (c) U.S. provisional application 61/759,231 titled “Methods and Systems for Determining a Correlation Between Patient Actions and Symptoms of a Disease,” filed Jan. 31, 2013. The systems and methods disclosed in the above-listed applications are practiced by querying a database to determine whether the database includes a correlation between a particular action and a particular symptom for the particular disease. The correlation can be based on a plurality of patient datasets collected from patient data received from a plurality of patients over time. In response to determining that the database includes a correlation between the particular action and the particular symptom, the computing device may send an indication of the correlation to the individual patient. The above-listed applications are owned by Curelator, Inc., and this application incorporates the entire contents of the above-listed applications by reference.

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that aspects of the present disclosure, as generally described herein, can be arranged, substituted, combined, separated, and designed in a wide variety of different configurations, all of which are explicitly contemplated herein.

### BRIEF DESCRIPTION OF THE FIGURES

FIG. 1 shows a first method employed in various example embodiments of the systems and methods for increasing patient engagement and patient persistence for a digital health management application disclosed and described herein.

FIG. 2 shows a chart that illustrates expected patient compliance with entering tracking data into a digital health management application.

FIG. 3 shows an example computing device configured to execute the features and functions of the systems and methods for increasing patient engagement and patient persistence for a digital health management application disclosed and described herein.

FIG. 4 shows an example method that includes providing an indication of a particular NAF according to some embodiments.

### DETAILED DESCRIPTION OF EXAMPLE EMBODIMENTS SHOWN IN THE FIGURES

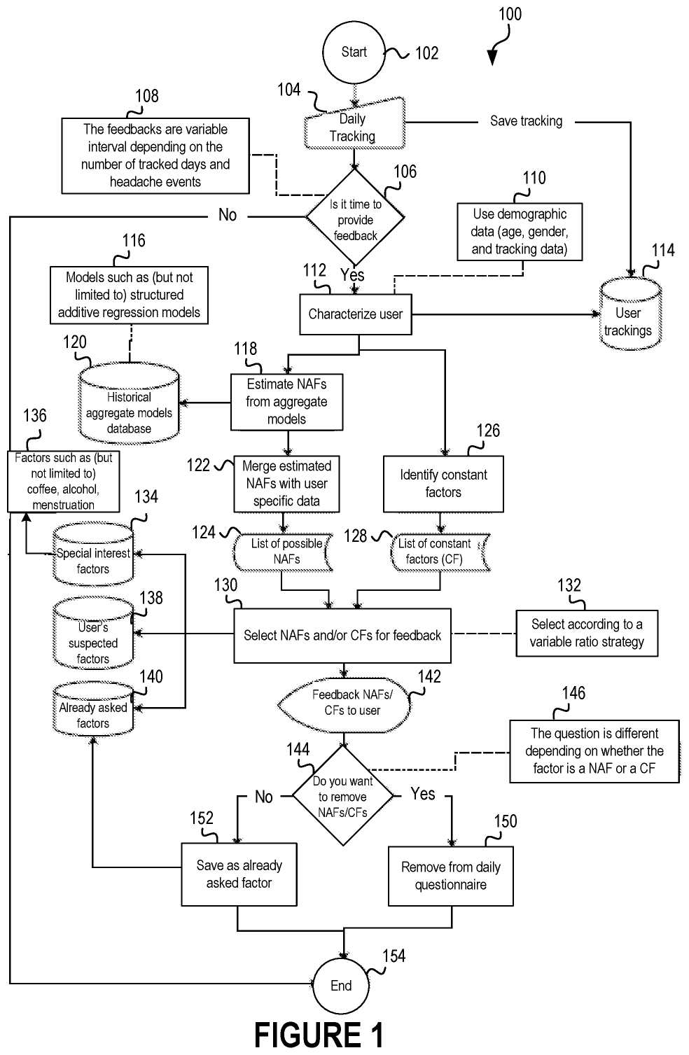

FIG. 1 shows a first method 100 employed in various example embodiments of the systems and methods for increasing patient engagement and patient persistence for a digital health management application disclosed herein. Although certain functional blocks of method 100 are illustrated in a sequential order, these blocks may in some instances be performed in parallel, and/or in a different order than those described herein. Also, the various blocks may be combined into fewer blocks, divided into additional blocks, and/or removed based on the desired implementation.

Method 100 begins at starting point 102, where a smartphone or other computing device (referred to herein generically as a computing device) launches a health management application (referred to herein generically as the application). In some embodiments, the computing device launches the application in response to a request received (e.g., via a user interface) from a patient, e.g., a smartphone launches the application in response to the patient selecting the application via an icon in the user interface of the computing device.

In some embodiments, the application (individually or in combination with one or more additional applications) is configured to perform one or more features comprising: (i) monitor and collect tracking data about the patient's disease symptoms, disease factors, and/or disease triggers; (ii) aggregate and/or organize information regarding the patient's disease symptoms, disease factors, and/or disease triggers; (iii) determine statistical associations and/or correlations between (iii-a) the patient's disease symptoms and (iii-b) the patient's disease factors and/or disease triggers; (iv) identify the patient's disease factors and/or disease triggers that are most likely to cause the patient to experience a particular disease symptom and/or prevent the patient from experiencing a particular disease symptom; (v) inform the patient when they are at a high risk of experiencing a particular disease symptom; (vi) show the patient the impact that his or her conduct or environment might have on the patient's likelihood of experiencing the particular disease symptom; (vii) generate and send messages (e.g., emails, text messages, or other types of messages or notifications) to and/or display such messages to a patient reminding the patient to (a) avoid engaging in certain activities that have been identified as increasing a likelihood of experiencing a disease symptom (i.e., disease triggers, as described herein), or (b) to engage in certain activities that have been identified as reducing the likelihood that the patient will experience the particular disease symptom (i.e., protectors, as described herein); (viii) inform the patient that some disease factors are no-association factors (NAFs) that neither increase the likelihood that the patient will experience the particular disease symptom (i.e., the NAF is not a disease trigger) nor reduce the likelihood that the patient will experience the particular disease symptom (i.e., the NAF is not a protector)); and/or (ix) inform the patient that some disease factors are invariable factors, referred to herein as “IFs”.

For example, in some embodiments, the application can be any of the health management applications disclosed or described in any of: (i) U.S. application Ser. No. 15/502,087 titled “Chronic Disease Discovery and Management System,” filed on Feb. 6, 2017; (ii) PCT application PCT/US15/43945 titled “Chronic Disease Discovery and Management System,” filed on Aug. 6, 2015; (iii) U.S. provisional application 62/034,408 titled “Disease Symptom Trigger Map,” filed on Aug. 7, 2014; (iv) U.S. provisional application 62/120,534 titled “Chronic Disease Management System,” filed on Feb. 25, 2015; (v) U.S. provisional application 62/139,291 titled “Chronic Disease Discovery and Management System,” filed on Mar. 27, 2015; (vi) U.S. provisional application 62/148,130 titled “Chronic Disease Discovery and Management System,” filed on Apr. 15, 2015; (vii) U.S. provisional application 62/172,594 titled “Chronic Disease Discovery and Management System,” filed on Jun. 8, 2015; (viii) PCT application PCT/US14/13894 titled “Methods and Systems for Determining a Correlation Between Patient Actions and Symptoms of a Disease,” filed on Jan. 30, 2014; (ix) U.S. provisional application 61/860,893 titled “Methods and Systems for Determining a Correlation Between Patient Actions and Symptoms of a Disease,” filed on Jul. 31, 2013; (x) U.S. provisional application 61/762,033 titled “Methods and Systems for Determining a Correlation Between Patient Actions and Symptoms of a Disease,” filed on Feb. 7, 2013; and/or (xi) U.S. provisional application 61/759,231 titled “Methods and Systems for Determining a Correlation Between Patient Actions and Symptoms of a Disease,” filed Jan. 31, 2013.

In some embodiments, for a specific patient, (i) the disease symptom is a migraine headache, (ii) disease factors are any event, exposure, action, or conduct related to and/or performed or experienced by the patient that has the potential to influence, affect, or cause the patient to experience a migraine headache, or in some cases, prevent the patient from experiencing a migraine headache, (iii) a disease trigger is a disease factor that has been determined, for example through statistical analyses or other methods, to have a sufficiently strong association with increasing the likelihood that the patient will experience a migraine headache, (iv) a disease protector is a disease factor that has been determined, for example through statistical analyses or other methods, to have a sufficiently strong association with reducing the likelihood that the patient will experience a migraine headache, (v) an NAF is a disease factor that has been determined, for example through statistical analyses or other methods, to not have any sufficiently strong association with increasing or reducing the likelihood that the patient will experience a migraine headache, and (vi) an invariable disease factor (IF) is a disease factor that does not vary (remains constant) through the tracking data. However, some embodiments could be used with other diseases or ailments, including but not limited to diseases and ailments that tend to be episodic in nature, e.g., epilepsy, irritable bowel syndrome, hay fever, depression, diabetes, and other diseases and/or ailments.

Next, method 100 advances to block 104, where the application receives tracking data from the patient and saves the tracking data into tracking database 114. In some embodiments, the application receives at least some tracking data from a patient via the patient entering the tracking data into a user interface of or associated with the application. In some embodiments, the application receives at least some tracking data from one or more monitors or sensors worn or used by the patient.

In addition to tracking data for the individual patient, in some embodiments, the tracking database 114 may additionally store at least a portion of the aggregate patient tracking data from at least a portion of a larger patient population. In some embodiments, block 104 includes receiving tracking data from the patient once a day and/or perhaps multiple times per day. In some embodiments block 104 can include data collected from external devices, like (but not limited to) a heart rate monitor, glucose monitor, smartwatch, fitness tracker, or other devices or applications. In some embodiments, the health management application tracks the frequency with which it receives tracking data from the patient. In some embodiments, the tracking information includes information about (i) whether and the extent to which the patient has experienced any disease symptoms, and (ii) whether and the extent to which the patient has been exposed to any disease factors.

Next, method 100 advances to block 106, where the application determines whether it should provide any feedback to the patient. In some embodiments, the feedback comprises informing the patient that a particular disease factor is an NAF or is invariable (IF). In some embodiments, the feedback is delivered to the patient according to a variable “reward” schedule. For example, in some embodiments, the feedback varies according to one or more intervals depending at least in part on (i) the number of days that the patient has been entering tracking data into the application, (ii) the number of disease symptoms (e.g., the number of migraine headaches) included in the tracking data the patient has entered into the application within a defined timeframe (e.g., days, weeks, months, etc.) or perhaps since the patient first started entering tracking data into the application, (iii) the frequency with which the patient has entered tracking data (e.g., once hourly, once every few hours, a few times a day, once a day, once every few days, once a week, etc.), and/or (iv) the volume of tracking data the patient has entered into the application (e.g., tracking data for a single disease factor, a few disease factors, or many disease factors). In some embodiments, the feedback is provided at a variable interval depending on the number of days the patient has been entering tracking data into the application and the number of disease symptoms (e.g., migraine headache events) the patient has experienced since the patient began entering tracking data into the application, as shown in comment block 108.

In some embodiments, determining whether it is time for the application to provide feedback to the patient at block 106 sometimes includes providing feedback more often when the patient has been entering less tracking data, and entering the tracking data less often, than a threshold amount or frequency. In operation, providing feedback more often when the patient has been entering less tracking data less often than the threshold is expected to spur the patient to enter more tracking data more often because, by receiving the feedback, the patient recognizes that entering the tracking data is providing a tangible benefit to the patient.

In some embodiments, determining whether it is time for the application to provide feedback to the patient at block 106 additionally or alternatively includes providing feedback less often when the patient has been entering more tracking data, and entering the tracking data more often, than a threshold amount or frequency. In operation, providing feedback less often when the patient has been entering more tracking data more often than the threshold allows the application to save feedback for when the patient starts to enter less tracking data less often. However, in some embodiments where the patient has been entering more tracking data more often, the application has more feedback to provide to the patient, and thus, in such embodiments, the application provides more feedback more often when the patient is providing more tracking data more often to encourage the patient to continue entering tracking data on a regular basis.

In some embodiments, determining whether it is time for the application to provide feedback to the patient at block 106 additionally or alternatively includes determining whether the application has more than a threshold number NAFs to provide to the patient. If the application has more than a threshold number of NAFs (e.g., more than 3, 5, 7, etc. NAFs) to provide to the patient, then at block 106, the application may determine that it is time to provide feedback about NAFs to the patient. And if the application has less than the threshold number of NAFs (e.g., less than 3 or 5 NAFs) to provide to the patient, then at block 106, the application may determine that it is not time to provide feedback about NAFs to the patient until the application has more confirmed NAFs. In certain embodiments whether or not NAFs are provided will also depend on the significance of the NAFs to the patient, e.g., where the NAF is a behavior or condition occurring at a frequency greater than a threshold value.

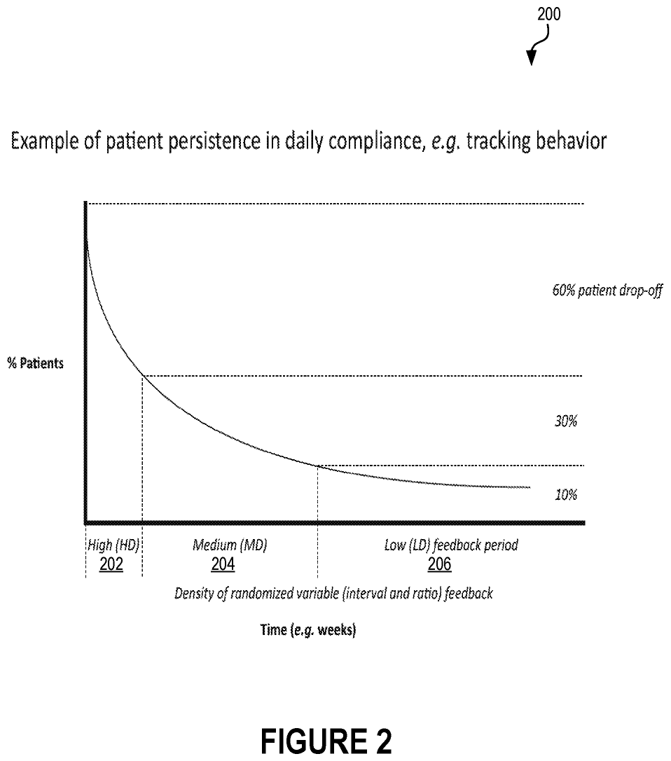

In some embodiments, determining whether it is time for the application to provide feedback to the patient at block 106 is based at least in part on how long the patient has been entering tracking data. For example, FIG. 2 shows an example chart 200 illustrating typical patient persistence in entering tracking data on a daily basis for a typical patient population over time. As shown in FIG. 2, about 60% of patients tend to stop entering tracking data on a daily basis within about a week or two after first starting to use the application, about 90% of patients tend to stop entering tracking data on a daily basis after a few weeks, and only about 7-10% of patients tend to continue entering tracking data on a daily basis after a few months. To encourage more patients to enter tracking data on a daily basis for longer time periods, in some embodiments, the application (i) provides a high density of feedback (i.e., more feedback more often) during a high density period 202, e.g., 1-2 NAFs every day or two for the first few weeks after the patient first starts entering tracking data into the application, (ii) provides a medium density of feedback (i.e., some feedback somewhat often) during a medium density period 204, e.g., a few NAFs every few days starting a few weeks after the patient first started entering tracking data, and (iii) provides a low density of feedback during a low density feedback period 206, e.g., 1-2 NAFs each week starting a few months after the patient first started entering tracking data.

By giving patients more feedback early, such embodiments condition patients to provide feedback on a daily basis by both (i) showing the patient that entering data provides tangible benefits to managing their disease symptoms and (ii) reducing the number of disease factors that the patients need to track (because the patient no longer needs to enter tracking data for NAFs), thereby making daily tracking easier over time. Also, by giving patients more feedback early, patients should tend to stay engaged with the application for longer, thereby enabling the application to collect more tracking data from the patient, and thereby collect sufficient tracking data to confirm one or more disease triggers and disease protectors before the patient would otherwise begin to lose interest in entering tracking data into the application on a daily basis. And when the patient receives confirmation of disease triggers and disease protectors, the patient is more likely to continue entering tracking data on a daily basis (or at least on a reasonably regular basis) to hopefully receive confirmation of more disease triggers and disease protectors that the patient can use to better manage his or her disease symptoms.

In some embodiments, determining whether it is time for the application to provide feedback to the patient at block 106 includes determining whether to provide feedback to the patient based on any combination of (i) how long the patient has been entering tracking data, e.g., according to a high density 202, medium density 204, and 206 low density phased approach, (ii) whether the application has stored more or fewer than a threshold number of NAFs, (iii) whether the patient has been entering less tracking data, and entering the tracking data less often, and/or (iv) whether the patient has been entering more tracking data, and entering the tracking data more often.

In some embodiments, determining whether it is time for the application to provide feedback to the patient at block 106 includes one or more of (i) only providing feedback on days when the patient has entered tracking data that day, (ii) only providing feedback on a day after the patient has entered tracking data for at least two consecutive days, (iii) only providing feedback on a day after the patient has entered tracking data for three or more consecutive days, and/or (iv) not providing feedback on a day when the patient has not entered tracking data.

If at block 106, the application decides that it is not time to provide feedback to the patient, method 100 advances to block 154 and ends.

But if at block 106, the application decides that it is time to provide feedback to the patient, method 100 advances to block 112, where the application characterizes the patient. In some embodiments, characterizing the patient includes using demographic data (e.g., age, gender, and other demographic data) as well as tracking data that the patient has previously entered into the application, including for example, tracking data stored in database 114.

In some embodiments, characterizing the patient at block 112 is optional. In some embodiments, the application may have previously characterized the patient such that an additional characterization at block 112 would be redundant or otherwise unnecessary.

Next, method 100 generates (i) a list of possible NAFs and (ii) a list of invariable factors (IFs).

To generate the list of possible NAFs, the application (i) estimates the NAFs from aggregate models at block 118, (ii) merges estimated NAFs with patient specific data at block 122, and (iii) generates the list of possible NAFs at block 124. In some embodiments, estimating the NAFs from aggregate models at block 118 includes the application (individually or in combination with a separate server system) estimating one or more NAFs from aggregate patient tracking data. In some embodiments, the application may additionally or alternatively estimate at least some NAFs from patient specific tracking data without necessarily considering the aggregate patient tracking data. In some embodiments, estimating one or more NAFs from aggregate patient tracking data comprises estimating one or more NAFs based on historical aggregate patient tracking data from a population of patients over time. In some embodiments, the historical aggregate patient tracking data is stored in a historical aggregate models database 120. In some embodiments, the historical aggregate models include, but are not limited to, structured additive regressive models, as indicated by comment block 116. The generated list of possible NAFs may be referred to as a list of population NAFs. Or, where additional personal disease factors are included in the list, the generated list of possible NAFs may be referred to as a list of global NAFs. To generate the list of invariable factors (IFs), method 100 (i) identifies invariable (constant) factors at block 126, and then (ii) generates the list of constant factors at block 128.

Next, method 100 advances to block 130, which includes selecting NAFs and/or IFs for feedback to the patient. In some embodiments, selecting NAFs and/or IFs for feedback to the patient includes selecting NAFs and/or IFs from one or more of (i) special interest disease factors stored in a special interest disease factors database 134, including but not limited to special disease factors such as coffee, alcohol, menstruation, or other special interest disease factors, as indicated in comment block 136, (ii) disease factors suspected by the patient stored in a patient-suspected disease factors database 138, and/or (iii) “already asked” disease factors stored in an “already asked” disease factors database 140. In some embodiments, the special interest disease factors stored in the special interest disease factors database 134 are specified by the application, perhaps based on common disease factors from the aggregate patient tracking data (but need not be). In some embodiments, the patient-suspected disease factors stored in the patient-suspected disease factors database 138 include suspected personal disease factors specified by the patient and may include disease factors that the patient believes to be either disease triggers or disease protectors. For example, the patient may select the suspected personal disease factors from a list of disease factors for tracking by the health management application. The selected personal disease factors may intersect with a set of population disease factors associated with aggregate population data, but also may include one or more disease factors not included in the set of population disease factors. The personal disease factors and population disease may collectively be referred to as global disease factors. Personal disease factors determined to have no effect on the patient may be referred to as personal NAFs. And in some embodiments, the “already asked” disease factors stored in the “already asked” disease factors database 140 include disease factors that have been saved in the “already asked” disease factors database 140 by the patient in response to prompting by the application at block 152.

In some embodiments, selecting NAFs and/or IFs for feedback to the patient includes selecting NAFs and/or IFs according to a variable ratio strategy, as indicated in comment block 132. In some embodiments, the variable ratio strategy is based at least in part on the high density 202, medium density 204, and 206 low density periods shown in FIG. 2. For example, if the high density period 202 represents about 60% of aggregate drop-off in patient engagement, then about 60% of the NAFs are allocated to be provided to the patient as feedback during the high density period 202. And if the medium density period 204 represents about another 30% aggregate drop-off in patient engagement, then about another 30% of the NAFs are allocated to be provided to the patient as feedback during the medium density period 204. And if the low density period 206 represents about the last 10% aggregate drop-off in patent engagement, then about another 10% of the NAFs are allocated to be provided to the patient during the low density period 206.

Rather than feeding back NAFs proportional to the periods of aggregate drop-off in patient engagement, some embodiments may feed back more NAFs to patients sooner. For example, in some embodiments, some embodiments may allocate about 70% of the NAFs for feedback during the high density 202 period, about 20% of the NAFs for feedback during the medium density period 204, and about 10% of the NAFs for feedback during the low density period 206. Other variable ratio scenarios could be used, too.

In this manner, method 100 provides more feedback to patients sooner to encourage patients to continue to enter tracking data into the application on a daily basis for longer, thereby collecting more tracking data and increasing the likelihood that the application will be able to confirm one or more disease triggers and disease protectors for the patient, which should, in turn, encourage the patient to continue to enter more tracking data in to the application in hopes of learning more confirmed disease triggers and disease protectors.

Further, as patients continue to enter tracking data into the application, the likelihood of the application confirming one or more disease triggers and/or disease protectors increases. So, in operation, the NAFs are best utilized sooner rather than later to encourage patients to enter sufficient data to confirm one or more disease triggers and/or disease protectors.

After selecting the NAFs and/or IFs for feedback at block 130, method 100 advances to block 142, which includes providing feedback (e.g., one or more messages) to the patient, where the feedback message(s) identifies one or more of the selected NAFs and/or IFs. In operation, the message is different depending on whether the feedback provided to the patient at block 142 is related to an NAF or an IF, as indicated in comment block 146.

For example, in some embodiments, providing feedback to the patient regarding a selected NAF includes displaying a message via the user interface that states, for example, “Congratulations! Your daily data entry has been analyzed by Curelator to reveal that [selected NAF] is NOT associated with your migraine attacks. Do you want to keep tracking [selected NAF] or should we remove it from your daily tracking list?” In some embodiments, providing feedback to the patient regarding a selected IF includes displaying a message via the user interface that states, for example, “Your daily data entry has been analyzed by Curelator and we've detected that [selected IF] is invariable through your tracking data. Do you want to keep tracking [selected IF] or should we remove it from your daily tracking list?” Other feedback messages for NAFs and/or IFs could be used, too.

If at block 144, the application receives an indication from the patient that the patient does not wish to remove the NAF and/or IF presented in block 142 from the patient's list of tracked disease factors (e.g., via a graphical user interface input), then method 100 advances to block 152, which includes saving the NAF and/or IF presented in block 142 to the “already asked” factors database 140. Then, method 100 advances to block 154 where method 154 ends. In some embodiments, block 152 might be performed any time feedback is provided to a patient at block 144, whether or not the patient indicates that he or she wants to remove an NAF or IF from the tracking list. In this fashion, all NAFs or IFs provided to the patient can be saved as “already asked” factors in “already asked” factors database 140.

But if at block 144, the application receives an indication from the patient that the patient wishes to remove the NAF and/or IF presented in block 142 from the patient's list of disease factors to track (e.g., via a graphical user interface input), then method 100 advances to block 152, which includes removing the NAF and/or IF presented in block 142 from the patient's list of disease factors to track. Once the NAF and/or IF presented in block 142 has been removed from the list of disease factors to track, the patient has fewer disease factors to track, and thus, less tracking data to enter on a daily basis. Then, method 100 advances to block 154 where method 154 ends.

In some embodiments, the patient will receive feedback of more than one NAFs and/or IFs, in which case the user interface may allow users to select one, some or all factors to remove from the tracked factors.

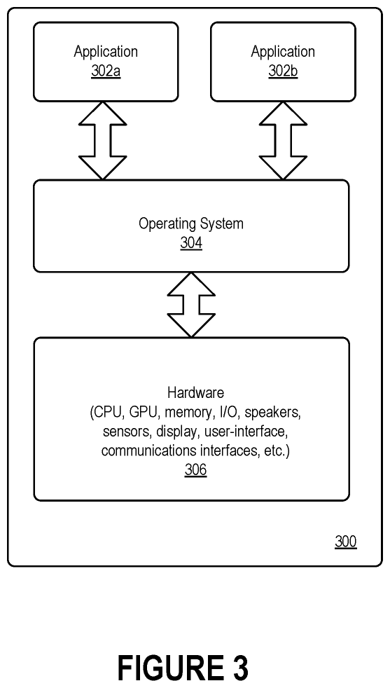

FIG. 3 shows an example computing device 300 configured to execute one or more (or all) of the features and functions of the early feedback methods disclosed and described herein. The computing device 300 can be a smartphone, tablet, desktop or laptop computer, or any other type of computing device with the capability of generating, gathering, and/or presenting data disclosed and described herein to a patient as well as performing any ancillary functions that may be required for effective implementation of the feedback methods disclosed and described herein.

Computing device 300 includes hardware 306 comprising: (i) one or more processors (e.g., a central processing unit(s) or CPU(s) and/or graphics processing unit(s) or GPU(s)); (ii) tangible non-transitory computer readable memory; (iii) input/output components (e.g., speaker(s), sensor(s), display(s), headphone jack(s) or other interfaces); and (iv) communications interfaces (wireless and/or wired). The hardware 306 components of the computing device 302 are configured to run software, including an operating system 304 (or similar) and one or more applications 302a, 302b (or similar) as is known in the computing arts. One or more of the applications 302a and 302b may correspond to computer-executable program code that, when executed by the one or more processors, cause the computing device 300 to perform one or more of the functions and features described herein, including but not limited to any (or all) of the features and functions of method 100, as well as any other ancillary features and functions known to persons of ordinary skill in the computing arts that may be required or at least desired for effective implementation of the features and functions of method 100, even if such ancillary features and/or functions are not expressly disclosed herein.

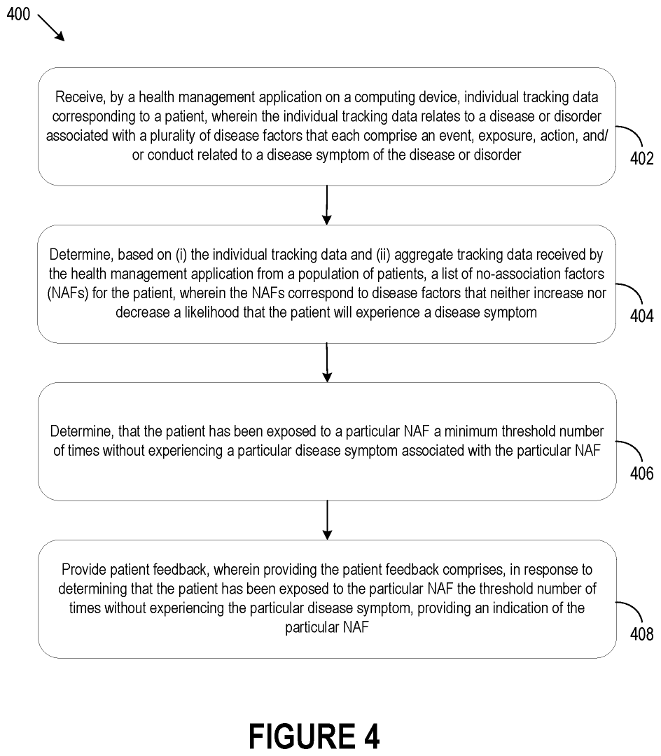

FIG. 4 shows an example method 400 that includes providing an indication of a particular NAF according to some embodiments. Although the blocks are illustrated in a sequential order, these blocks may in some instances be performed in parallel, and/or in a different order than those described herein. Also, the various blocks may be combined into fewer blocks, divided into additional blocks, and/or removed based on the desired implementation. Additionally, the example method 400 shows a client device performing some steps and a server performing other steps, but in alternative embodiments, some of the steps performed by a client in example method 400 may be performed by a server and vice versa.

Also, in method 400, each block may represent a module, a segment, or a portion of program code, which includes one or more instructions executable by a processor or computing device for implementing specific logical functions or steps in the method. The program code may be stored on any type of computer readable medium or memory, for example, such as a storage device including a disk or hard drive or other type of memory, such as flash memory or the like. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), and/or flash memory for example. The computer readable media may also be any other volatile or non-volatile storage systems. The computer readable medium may be considered a computer readable storage medium, for example, or a tangible storage device.

Example method 400 begins at block 402. Block 402 can be performed to receive, by a health management application on a computing device, individual tracking data corresponding to a patient, wherein the individual tracking data relates to a disease or disorder associated with a plurality of disease factors that each includes an event, exposure, action, and/or conduct related to a disease symptom of the disease or disorder. In operation, the computing device can be the same or similar to any of the computing devices disclosed herein.

Block 404 of method 400 can be performed to determine, based on (i) the individual tracking data and (ii) aggregate tracking data received by the health management application from a population of patients, a list of no-association factors (NAFs) for the patient, wherein the NAFs correspond to disease factors that neither increase nor decrease a likelihood that the patient will experience a disease symptom. The list of NAFs may be a list of global NAFs determined from a set of suspected population disease factors and a set of suspected personal disease factors. In some examples, the list may initially include population NAFs, and later include suspected personal NAFs selected by the patient.

In some examples, the disease or disorder may include a migraine, the disease symptom may include a migraine headache, and the disease factors include events, exposures, actions, or conduct associated with the patient experiencing a migraine headache. However, other diseases or disorders and corresponding disease symptoms and disease factors are possible as well.

Block 406 of method 400 can be performed to determine that the patient has been exposed to a particular NAF a minimum threshold number of times without experiencing a particular disease symptom associated with the particular NAF. For example, the minimum threshold number of exposures can be two exposures. Receiving individual tracking data may allow the health management application to determine that the patient has been exposed to a particular NAF at least the minimum threshold number of times.

Block 408 of method 400 can be performed to provide patient feedback, wherein providing the patient feedback comprises, in response to determining that the patient has been exposed to the particular NAF the threshold number of times without experiencing the particular disease symptom, providing an indication of the particular NAF. As described above, providing an indication of an NAF may encourage patient engagement by allowing the patient to input less data into the health management application, as further described below. Further, as described above with respect to FIG. 1, patient feedback may be provided in the form of indications of disease factors determined to be NAFs or IFs and options to remove the NAFs or IFs from a tracking list of disease factors.

In some examples, performing block 408 to provide the indication of the particular NAF includes selecting the particular NAF from the list of NAFs based on aggregate patient data and analysis of the population of patients with the health management application. For example, selecting the particular NAF may include determining a disease factor commonly suspected to be a disease trigger within a population of patients, but that is statistically likely an NAF within the population (e.g., based on all or substantially all past patient tracking data received from patients in the population). In such examples, the selected NAF may be a population NAF determined from a set of suspected population disease factors.

In an example embodiment, performing block 408 to provide the indication of the particular NAF may be performed as part of an intermittent feedback schedule in which indications of NAFs are provided to the patient in a random fashion, or in a random fashion but subject to constraints. For example, variability in the likelihood of providing feedback at a given time might be determined by a frequency that the health management application receives the individual tracking data, a number of NAFs in the list of NAFs, a volume of data received from the patient by the health management application, and other factors.

In another example embodiment, performing block 408 to provide the indication of the particular NAF includes providing an option to remove the particular NAF from a tracking list of disease factors, wherein removing the particular NAF from the tracking list corresponds to reducing an amount of individual tracking data requested by the health management application.

In such examples, method 400 may further include receiving an indication not to remove the particular NAF from the tracking list of disease factors, and, responsive to receiving the indication, saving the NAF to a database that corresponds to NAFs which have been provided as feedback to the patient and have not been selected for removal from the tracking list of disease factors. The database can be, for example, “already asked” disease factors database 140. Method 400 may further include receiving further individual tracking data corresponding to the particular NAF, confirming, based on the further individual tracking data, that the particular NAF corresponds to a disease factor that neither increases nor decreases a likelihood that the patient will experience a particular disease symptom, and providing, based on the confirmation, a second option to remove the particular NAF from the tracking list of disease factors. Confirming that the particular NAF corresponds to a disease factor that neither increases nor decreases a likelihood that the patient will experience a particular disease symptom may include receiving a threshold number of days receiving the further individual tracking data or a threshold number of detected instances of a disease symptom. For example, receiving 10 days of individual tracking data and/or detecting instances of a disease symptom may form a basis for confirming that the particular NAF corresponds to a disease factor that neither increases nor decreases a likelihood that the patient will experience a particular disease symptom.

In alternative embodiments, method 400 may further include receiving an indication to remove the particular NAF from the list of disease factors. In these examples, method 400 may further include, responsive to receiving the indication, removing the particular NAF from a daily questionnaire associated with the individual tracking data. In this fashion, the patient may enter less individual tracking data.

In an example embodiment, method 400 may further include determining a frequency with which the health management application received the individual tracking data. For example, the health management application may determine that the patient has entered data a certain number of times on a daily basis, weekly basis, monthly basis, or at another measure for how frequently the patient inputs the individual tracking data. The frequency may be indicative of a level of patient engagement with the health management application. In such examples, method 400 may further include comparing the frequency to a threshold frequency, and performing block 408 to provide the patient feedback can include providing the indication of the particular NAF based on determining that the frequency is above the threshold frequency. Providing the indication of the NAF when patient data input is above the threshold frequency may spur patient engagement by rewarding consistently high patient engagement. In other examples, providing the patient feedback may be performed responsive to determining that the frequency falls below the threshold frequency, and in this fashion may facilitate patient retention for patients that provide consistently low patient engagement.

In another example embodiment, method 400 may further include determining a number of days that the health management application received the individual tracking data, and comparing the number of days to a threshold number. In such examples, performing block 408 to provide the patient feedback may include providing the indication of the particular NAF based on determining that the number of days is above the threshold number. The threshold number of days can be two days. Providing feedback after the threshold number of days may allow the health management application to store several NAFs for future feedback. In other examples, providing the patient feedback may include providing the indication of the particular NAF based on determining that the number of days is below the threshold number. By providing feedback within a few days of starting to receive individual tracking data, the health management application can spur additional engagement from the patient at an early stage of patient engagement.

In another example embodiment, method 400 may include determining, based on the individual tracking data, an invariable disease factor (IF) of the patient, wherein the IF corresponds to a disease factor for which the tracking data does not change over time. In such examples, performing block 408 to provide the patient feedback can include providing an indication of the IF and an option to remove the IF from a tracking list of disease factors, wherein removing the IF from the tracking list corresponds to reducing an amount of individual tracking data requested by the health management application. Providing an option to remove the IF from the tracking list may allow a user to provide less tracking data to the health management application, and thus may serve as an incentive to provide more tracking data.

In another example embodiment, method 400 may include receiving, by the health management application, an indication of suspected personal disease factors, wherein the suspected personal disease factors correspond to disease factors that the patient selected as likely disease triggers or disease protectors. For example, the health management application may provide the user with an option to select disease factors for a given disease or disorder that the patient considers a likely disease trigger or protector. In such examples, prior to providing the indication of the particular NAF, method 400 can include prioritizing the particular NAF for patient feedback based on determining that the particular NAF corresponds to a suspected personal disease factor. In this scenario, the particular NAF may be referred to as a personal NAF. In such examples, performing block 408 to provide the patient feedback can include providing the indication of the particular NAF based on determining that the particular NAF corresponds to a suspected personal disease factor.

In another example embodiment, method 400 includes determining a plurality of population disease factors from the aggregate tracking data received by the health management application, and, prior to providing the indication of the particular NAF, determining that the particular NAF does not correspond to a population disease factor of the plurality of population disease factors. In such examples, performing block 408 to provide the patient feedback may include providing the indication of the particular NAF based on determining that the particular NAF does not correspond to the population disease factor.

In another example embodiment, method 400 includes determining a total number of NAFs in the list of NAFs associated with the patient, comparing the number of NAFs to a threshold number of NAFs, and determining that the number of NAFs exceeds the threshold number of NAFs. In such examples, performing block 408 to provide the patient feedback may include providing the indication of the particular NAF based on determining that the number of NAFs exceeds the threshold number of NAFs. In this fashion, the health management application may provide patient feedback when there is an excess of feedback to provide.

While particular aspects and embodiments are disclosed herein, other aspects and embodiments will be apparent to those skilled in the art in view of the foregoing teaching. For example, while the embodiments and examples are described with respect to migraine headaches, the disclosed systems and methods are not so limited and may be applicable to a broad range of disease symptoms and related disease factors and disease triggers. The various aspects and embodiments disclosed herein are for illustration purposes only and are not intended to be limiting, with the true scope and spirit being indicated by the following claims.

What is claimed is:

1. A method comprising:
receiving, by a health management application on a computing device, individual tracking data corresponding to a patient, wherein the individual tracking data relates to a disease or disorder associated with a plurality of disease factors that each comprise an event, exposure, action, and/or conduct related to a disease symptom of the disease or disorder;
determining, based on (i) the individual tracking data and (ii) aggregate tracking data received by the health management application from a population of patients, a list of no-association factors (NAFs) for the patient, wherein the NAFs correspond to disease factors that neither increase nor decrease a likelihood that the patient will experience a disease symptom;
determining that the patient has been exposed to a particular NAF a minimum threshold number of times without experiencing a particular disease symptom associated with the particular NAF; and
providing patient feedback, wherein providing the patient feedback comprises, in response to determining that the patient has been exposed to the particular NAF the threshold number of times without experiencing the particular disease symptom, providing an indication of the particular NAF.

2. The method of claim 1, further comprising:
determining a frequency with which the health management application received the individual tracking data; and
comparing the frequency to a threshold frequency,
wherein providing the patient feedback comprises providing the indication of the particular NAF based on determining that the frequency is above the threshold frequency.

3. The method of claim 1, further comprising:
determining a number of days that the health management application received the individual tracking data; and
comparing the number of days to a threshold number,
wherein providing the patient feedback comprises providing the indication of the particular NAF based on determining that the number of days is above the threshold number.

4. The method of claim 1, further comprising:
determining, based on the individual tracking data, an invariable disease factor (IF) of the patient, wherein the IF corresponds to a disease factor for which the tracking data does not change over time,
wherein providing the patient feedback comprises providing an indication of the IF and an option to remove the IF from a tracking list of disease factors, wherein removing the IF from the tracking list corresponds to reducing an amount of individual tracking data requested by the health management application.

5. The method of claim 1, further comprising:
receiving, by the health management application, an indication of suspected personal disease factors, wherein the suspected personal disease factors correspond to disease factors that the patient selected as likely disease triggers or disease protectors; and
prior to providing the indication of the particular NAF, prioritizing the particular NAF for patient feedback based on determining that the particular NAF corresponds to a suspected disease factor of personal interest,
wherein providing the patient feedback comprises providing the indication of the particular NAF based on determining that the particular NAF corresponds to a suspected disease factor.

6. The method of claim 1, further comprising:
determining a plurality of population disease factors from the aggregate tracking data received by the health management application; and
prior to providing the indication of the particular NAF, determining that the particular NAF does not correspond to a population disease factor of the plurality of population disease factors,
wherein providing the patient feedback comprises providing the indication of the particular NAF based on determining that the particular NAF does not correspond to the population disease factor.

7. The method of claim 1, wherein the disease or disorder comprises a migraine, wherein the disease symptom comprises a migraine headache, and wherein the disease factors comprise events, exposures, actions, or conduct associated with the patient experiencing a migraine headache.

8. The method of claim 1, wherein providing the indication of the particular NAF comprises selecting the particular NAF from the list of NAFs based on aggregate patient data and analysis of the population of patients with the health management application.

9. The method of claim 1, wherein providing the indication of the particular NAF comprises:
providing an option to remove the particular NAF from a tracking list of disease factors, wherein removing the particular NAF from the tracking list corresponds to reducing an amount of individual tracking data requested by the health management application.

10. The method of claim 9, further comprising:
receiving an indication not to remove the particular NAF from the tracking list of disease factors; and
responsive to receiving the indication, saving the NAF to a database that corresponds to NAFs which have been provided as feedback to the patient and have not been selected for removal from the tracking list of disease factors.

11. The method of claim 10, further comprising:
receiving further individual tracking data corresponding to the particular NAF;
confirming, based on the further individual tracking data, that the particular NAF corresponds to a disease factor that neither increases nor decreases a likelihood that the patient will experience a particular disease symptom; and
providing, based on the confirmation, a second option to remove the particular NAF from the tracking list of disease factors.

12. The method of claim 9, further comprising:
receiving an indication to remove the particular NAF from the list of disease factors; and
responsive to receiving the indication, removing the particular NAF from a daily questionnaire associated with the individual tracking data.

13. The method of claim 1, further comprising:
determining a total number of NAFs in the list of NAFs associated with the patient;
comparing the number of NAFs to a threshold number of NAFs; and
determining that the number of NAFs exceeds the threshold number of NAFs,
wherein providing the patient feedback comprises providing the indication of the particular NAF based on determining that the number of NAFs exceeds the threshold number of NAFs.

14. The method of claim 1, wherein providing the patient feedback is performed as part of an intermittent feedback schedule.

15. A non-transitory computer readable medium having computer-executable program code stored thereon that, when executed by one or more processors, causes performance of one or more functions, the functions comprising:
receiving, by a health management application on a computing device, individual tracking data corresponding to a patient, wherein the individual tracking data relates to a disease or disorder associated with a plurality of disease factors that each comprise an event, exposure, action, and/or conduct related to a disease symptom of the disease or disorder;
determining, based on (i) the individual tracking data and (ii) aggregate tracking data received by the health management application from a population of patients, a list of no-association factors (NAFs) for the patient, wherein the NAFs correspond to disease factors that neither increase nor decrease a likelihood that the patient will experience a disease symptom;
determining, that the patient has been exposed to a particular NAF a minimum threshold number of times without experiencing a particular disease symptom associated with the particular NAF; and
providing patient feedback, wherein providing the patient feedback comprises, in response to determining that the patient has been exposed to the particular NAF the threshold number of times without experiencing the particular disease symptom, providing an indication of the particular NAF.

16. The non-transitory computer readable medium of claim 15, wherein providing the indication of the particular NAF comprises:
providing an option to remove the particular NAF from a tracking list of disease factors, wherein removing the particular NAF from the tracking list corresponds to reducing an amount of individual tracking data requested by the health management application.

17. The non-transitory computer readable medium of claim 16, the functions further comprising:
receiving an indication not to remove the particular NAF from the tracking list of disease factors; and
responsive to receiving the indication, saving the NAF to a database that corresponds to NAFs which have been provided as feedback to the patient and have not been selected for removal from the tracking list of disease factors.

18. The non-transitory computer readable medium of claim 17, the functions further comprising:
receiving further individual tracking data corresponding to the particular NAF;
confirming, based on the further individual tracking data, that the particular NAF corresponds to a disease factor that neither increases nor decreases a likelihood that the patient will experience a particular disease symptom; and
providing, based on the confirmation, a second option to remove the particular NAF from the tracking list of disease factors.

19. The non-transitory computer readable medium of claim 15, the functions further comprising:
determining, based on the individual tracking data, an invariable disease factor (IF) of the patient, wherein the IF corresponds to a disease factor for which the tracking data does not change over time,
wherein providing the patient feedback comprises providing an indication of the IF and an option to remove the IF from a tracking list of disease factors, wherein removing the IF from the tracking list corresponds to reducing an amount of individual tracking data requested by the health management application.

20. The non-transitory computer readable medium of claim 15, the functions further comprising:
receiving, by the health management application, an indication of suspected personal disease factors, wherein the suspected personal disease factors correspond to disease factors that the patient selected as likely disease triggers or disease protectors; and
prior to providing the indication of the particular NAF, prioritizing the particular NAF for patient feedback based on determining that the particular NAF corresponds to a suspected disease factor of personal interest,
wherein providing the patient feedback comprises providing the indication of the particular NAF based on determining that the particular NAF corresponds to a suspected disease factor.
